(19) United States
(12) Patent Application Publication
(10) Pub. No.: US 2013/0110245 A1
(43) Pub. Date: May 2, 2013

(54) IMPLANT HAVING A SANDWICH STRUCTURE, IMPLANT SYSTEM AND IMPLANT SUPPORT

(71) Applicants: Uwe Merklein, Aldenhoven (DE); Thomas Gries, Aachen (DE); Thorsten Deichmann, Aachen (DE); Annahit Arshi, Aachen (DE)

(72) Inventors: Uwe Merklein, Aldenhoven (DE); Thomas Gries, Aachen (DE); Thorsten Deichmann, Aachen (DE); Annahit Arshi, Aachen (DE)

(73) Assignee: 3T TEXTILTECHNOLOGIETRANSFER GMBH, Aachen (DE)

(21) Appl. No.: 13/638,134

(22) PCT Filed: Mar. 29, 2011
(86) PCT No.: PCT/DE2011/000342

§ 371 Date: Nov. 9, 2012

(30) Foreign Application Priority Data
Mar. 30, 2010 (DE) 10 2010 013 485.6
Oct. 19, 2010 (DE) 10 2010 048 710.4

(51) Int. Cl. A61F 2/44

(52) U.S. Cl. CPC A61F 2/442
USPC 623/17.16

(57) ABSTRACT

The treatment of painful intervertebral disc diseases attempts to counteract degeneration. On the one hand artificial nucleus pulposus prostheses are being developed, and on the other hand regenerative therapy methods using biomaterials in the intervertebral disc are being pursued. In both cases, massive herniation of the materials may occur as a result of existing annulus defects or annulus defects which have been enlarged because of surgical access. In light of this, the reliable closure and the regeneration of the annulus fibrosus are an important prerequisite and further development for treating herniated intervertebral discs and degenerative herniated intervertebral discs. According to the invention, an implant having a sandwich structure is used, wherein a reinforcing structure is arranged between a two-layer non-woven and the non-woven comprises in particular a biocompatible material.

The invention relates to an implant, in particular for promoting a biological closure of the annulus fibrosus of the intervertebral disc, having a sandwich structure, an implant system with the implant and a deployment instrument associated with the implant, and a package packing this.

Age- or injury-related degeneration of the intervertebral disc is the most frequent cause of back pain. Almost one third of the population over 40 years of age shows signs of this intervertebral disc degeneration (Boden, S D, D O Davis, T S Dina, N J Patronas, and S W Wiesel. “Abnormal Magnetic-Resonance Scans of the Lumbar Spine in Asymptomatic Subjects. A Prospective Investigation.” J Bone Joint Surg Am 72, no. 3 (1990): 403-8, Wiesel S W et al., Spine. 1984, 9:549-51). Degenerative diseases of the intervertebral disc therefore constitute a medical, social and economic problem which is to be taken seriously. The treatment of the degenerative disease of the intervertebral disc depends on the degree of severity (intervertebral disc protrusion or herniated intervertebral disc) and on the symptoms (radiating pain in the extremities, back pain, paraesthesia and paralysis phenomena) and range from the administering of analgesics, remedial gymnastic and physiotherapeutic measures to invasive surgical techniques.

Within the scope of degeneration, a herniated intervertebral disc is frequently an acute event, in which an emergence of intervertebral disc tissue into the vertebral canal occurs as a result of fissuring in the annulus fibrosis (AF), a ring-shaped fibrocartilage which encloses the jell-like nucleus pulposus (NP). Pain and neurological malfunctions occur owing to the effect of pressure onto the posterior longitudinal ligament and nerves. Frequent clinical symptoms are radiating pain, back pain, paraesthesia and paralysis phenomena.

In an operative intervention, the prolapse pressing onto the nerve is removed. In the further course of the disease, a progressive loss of height of the intervertebral disc and hence further degeneration phenomena can occur, which also affect the adjoining vertebral bodies. Finally, frequently only a reinforcing of the vertebral bodies, with associated restriction to freedom of movement remains as a therapeutic measure. The surgical care of a herniated intervertebral disc is the operation most frequently carried out on the vertebral column (Weinstein J N et al. Spine. 2006; 31 (23); 2707-2714).

In an intervertebral disc operation, the space-occupying tissue is removed from the spinal canal. However, the defect in the annulus fibrosus remains. Recurrent prolapses frequently occur through this opening. Under the hypothesis of being able to minimize the recurrence rate, the nucleus pulposus is removed as completely as possible during the operation. Nevertheless, approximately 10% of these patients must be operated on again within two years (Thome C. et al. J Neurosurg. Spine. 2005; 2(3); 271-278, Osterman H. et al. Spine 2003; 15; 28(6); 621-627).

For greater defects in the annulus fibrosus, the recurrence rate is even up to 21.5%. Using the current DRG system as the basis, the treatment costs which occur annually through recurrence operations amount to at least 200 Million Euro. The actual economic burden is likely to exceed the treatment costs many times over.

The cause of this is the substantially poorer clinical course of these patients. After a recurrence operation, up to 70% of them develop persistent backache, which results in repeated consultations, complex operations on the vertebral column and invalidity.

Innovative approaches in the treatment of painful intervertebral disc diseases attempt to counteract degeneration. On the one hand, artificial nucleus pulposus prostheses are being developed, and on the other hand, regenerative therapy methods using biomaterials in the intervertebral disc are being pursued (Di Martino A et al. Spine. 2005; 15; 30 (16 Suppl.); 16-22; Wilke H J et al. Eur. Spine. J. 2006; 15; Suppl. 15; 433-438).

In both cases, massive herniation of the used materials repeatedly occurred as a result of existing annulus defects and annulus defects which have been enlarged because of surgical access. In light of this, the reliable closure and the regeneration of the annulus fibrosus are an important prerequisite or further development for treating herniated intervertebral discs and degenerative intervertebral disc diseases.

There is currently no therapy strategy in clinical routine for the prevention of herniations. There are various annulus closure techniques with solid artificial materials (Barricaid®, Anulex®) in clinical trial. These materials are placed in front of the annulus defect as an artificial barrier device and thereby prevent the emergence of intervertebral disc tissue.

A disadvantage of these closures is, in particular, the risk of the migration of solid artificial materials which are used, which can lead to injuries to nerval structures. This can be accompanied by permanent neurological deficiencies such as paralysis phenomena. However, lack of integration into the surrounding body tissue, foreign body reactions and material wear are also to be named as disadvantages.

Currently, implants are not available either in clinical routine or in clinical trial which assist a biological repair and regeneration of the annulus (Hegewald A A et al. Front Biosci. 2008, 13:1507-1525).

The object of the invention to improve the prior art.

This problem is solved by an implant having a sandwich structure, in particular for promoting a biological closure of the annulus fibrosus of the intervertebral disc, wherein a reinforcing structure is arranged between a two-layer non-woven and the non-woven comprises in particular a biocompatible material.

Therefore, a soft, partially resorbable implant, based on textile, can be produced, in which a sufficient biomechanical primary stability is provided by the reinforcing structure and biological regeneration- and repair processes can be promoted, because these can connect readily with the surrounding tissue.

In particular, a promotion of the biological closure of the annulus fibrosus of the intervertebral disc can thus take place.

The concepts are to be explained below:

A “non-woven” (also designated as non-woven material) is a textile fabric of individual fibers. The term non-woven also includes felt, mostly produced from wool. In addition, non-woven includes all non-wovens or non-woven materials belonging in the standard DIN EN 29 092 (ISO 9092). The non-woven can consist of fibers lying loosely together, which are not yet connected with one another. The strength of a non-woven can rest substantially only on the adhesion inherent to the fibers, wherein this is also able to be influenced by reprocessing. The non-woven material which is predominantly used or the non-woven which is predominantly used can be further processed such that a strengthening is already effected. The fibers of a non-woven can lie in a haphazard manner with respect to one another. Both isotropic non-wovens and also anisotropic non-wovens are included. In the case of the isotropic non-wovens, the fibers have no preferred direction. In contrast to this, in the case of anisotropic non-wovens, the fibers are aligned more frequently in one direction than in others. Non-wovens include both haphazard non-wovens and also fiber-oriented non-wovens. The non-woven can be obtained both by spinning, needling and also by fulling.

“Two layer” is to be understood to mean, in particular, that two layers of non-woven are able to be laid one on another. This can take place on the one hand by two separate non-wovens, but this can also be achieved for example by folding one single-piece non-woven. Each non-woven layer can consist here of several doubled individual non-wovens.

The “reinforcing structure” is distinguished in particular in that it is not as deformable as the non-woven. The reinforcing structure, in particular in the case of knits, can be more flexible and/or more elastic than non-wovens. As the reinforcing structure can act in a stabilizing manner onto a cavity, herniations of the implant into the spinal canal can be avoided.

A “biocompatible material” is distinguished in that rejection reactions generally do not occur, or can be limited well medicinally.

In a further embodiment, the non-woven is resorbable or partially resorbable. Therefore, the non-woven can be replaced by substances produced naturally in the body.

“Resorbable” is understood to mean materials which in the course of two years are broken down or replaced by a body such that only five or less percent of the inserted material, which is to be resorbed, remains in the organism. “Partially resorbable” are substances of which within two years in a body only forty percent or less, but more than five percent, of the material remains in the body.

Through the resorbability or partial resorbability of the non-woven, biological regeneration- and repair processes are promoted, and connections to the surrounding tissue can be established. Therefore, the non-woven can be replaced by the body's own structures.

In order to fill a cavity with as little foreign material as possible, the non-woven can have a porosity of 30% to 99%, wherein the porosity of the non-woven lies in particular between 70% and 98%. The porosity can be determined here by the Solid Two Volume Fraction method. For this, the surface weight is determined according to ISO 9073-1, and the non-woven thickness is determined according to ISO 9073-2:1995. To determine the weight, the scales AE 163; Mettler Toledo, Ohio, USA is used. The thickness is determined by the Universal Micrometer, Frank Prüfgeräte GmbH, Birkenau.

In a further embodiment, the non-woven has one of the following materials or a combination of the materials: a polyglycolic acid, a polyactide acid, a polycaprolactone, a poly-3-hydroxybutyrate, a polydioxanone, copolymers thereof and various derivatives thereof. Through the use of these materials, biocompatible materials can be provided, which can be processed into non-wovens having sufficient porosity.

In order to assist the compatibility of the implant or the replacing of the non-woven structures by the body, the non-woven can be enriched with an organic material, in particular with a collagen, a hyaluronic acid, a glycosaminoglycan, a demineralized bone particle, a small intestine submucosa preparation and another organic preparation, which has a bioactive factor, in particular TGF-β, FGF, BMP and/or chemoattractants, in particular CXCL 10 and/or XCL 1.

In order to obtain the same advantage, the non-woven can be enriched with an inorganic material, in particular a hydroxyapatite, a calcium phosphate, a calcium sulfate, a metal and/or combinations thereof.

The abbreviation TGF stands here for Transforming Growth Factor, FGF for Fibroblast Growth Factor, BMP for Bone Morphogenetic Protein, CXCL for Chemokine (XC motif) Ligand, wherein XC describes the protein “motif” and XCL stands for Chemokine (C motif) ligand.

In order to facilitate the incorporation of the non-woven into the body, the non-woven can be cell-colonized, wherein the cell colonization takes place in particular with or without a prior in-vitro tissue maturation, wherein in particular cells with chondrogenic expression potential are able to be used.

Cells with chondrogenic expression potential are cells such as for example stem cells, which can convert in particular into cartilage cells, and autogenic and allogenic cartilage cells.

In a further embodiment, a pore size of the non-woven corresponds to one to four times a cell size, so that the cell colonization of the non-woven is optimized, wherein the pore size in particular has a diameter of between 9 μm and 2 mm. The determining of the pore size can take place with a microscope and the OPTIMAS software; Media Cybernetics, Silverstring, USA.

So that a stability can also be guaranteed after the breakdown of the non-woven, the reinforcing structure can be non-resorbable or poorly resorbable.

“Non-resorbable” materials are understood to mean materials which are broken down or replaced by a maximum of up to five percent within a stay of two years inside an organism. Poorly resorbable materials are understood to mean materials which are broken down or replaced by between five percent and 40 percent within a stay of two years in an organism.

In order to meet different mechanical stresses, the reinforcing structure can be configured as a knit, a knitted fabric, a weave, a network structure, a twisted yarn, a mesh or a multi-ply fabric.

The concepts are to be explained below:

A “knit” comprises a thread system with the formation of stitches. Knits belong to knitted goods. Both weft knits and warp knits are included.

A “knitted fabric” is a textile produced by means of knitting.

A “weave” comprises manually or mechanically produced products of weaving such as cloths, velvet, velours, plush, toweling or other textile flat weaves having at least two thread systems crossed at right-angles or almost at right-angles.

A “twisted yarn” is linear textile, which consists of yarn twisted from several or at least two wiry threads. For the production of the twisted yarn, methods such as the double wire twisting method, calibrating, winding twist method, ring twist method, step twist method, air eddying method or twist replacement method are used.

A “mesh” comprises a product of several strands of flexible material looped into one another, which is able to be produced in particular by means of braiding.

A “multi-ply fabric” is a special textile flat structure. It is distinguished by its good drapability, wherein the fibers are present substantially in stretched form. The multi-ply fabric can consist of several layers of fiber bundles or fiber strands arranged in parallel. The individual layers can differ in fiber orientation, wherein the fiber orientation is indicated in alignment with an angle to the production direction.

Both two-layer multi-ply fabrics, in which the alignment of the fibers can be for example 0° and 90°, or multi-layer multi-ply fabrics, with a layer alignment of 90°, −45°, 0°, +45°, which forms a four-layer multi-ply fabric, are included. The layers can be worked together with one another for better handling.

In order to influence mechanical characteristics such as tensile strength of the reinforcing structure, the reinforcing structure can have mono- and/or multi-filaments, which in particular form hernia nets.

“Filaments” here are fibers with a practically unlimited length. In particular, fibers with at least a length of 1000 mm are defined as a filament.

In order to provide an implant with different material characteristics, the reinforcing structure can have one of the following materials or a combination of the materials: a polyvinylidene fluoride, a polypropylene, a polyester, a polyamide, a silk material, a linen material, a metal or derivatives.

So that the reinforcing structure can be better worked into the organism and received by the latter, the reinforcing structure can be enriched with an organic material, in particular a collagen, a hyaluronic acid, a glycosaminoglycan, a demineralized bone particle, a small intestine submucosa preparation and another organic preparation which has a bioactive factor, in particular TGF-β, FGF, BMP and/or chemoattractants, in particular CXCL 10 and/or XCL 1.

In addition, the reinforcing structure can be enriched additionally or alternatively with an inorganic material, in particular a hydroxyapatite, a calcium phosphate, a calcium sulfate, a metal and/or combinations thereof.

In a further embodiment, the non-woven is produced aerodynamically or mechanically. Therefore, alternative non-wovens can be provided.

In the mechanical methods, a breaking up takes place up to a single fiber, by means of teasels, cards or installations operating my means of breaking-up rollers such as the MDTA 3, Uster Technologies, Uster, Switzerland. The single fibers are then doubled to a nap (the non-woven or non-woven material) with a desired thickness and surface weight and then further processed. For carding, the universal carding machines of Spinnbau GmbH, Bremen can be used, for example.

In the aerodynamic method, a fiber mass stream is delivered to an air stream. This air-fiber mixture flows through a screen. The fibers deposit themselves on the latter and form the non-woven. A compacting takes place by the through-flowing air. A collecting chamber, to which the fiber/air mixture is delivered, can be provided here with a widened cross-section, whereby the speed of flow of the fibers is reduced and the fibers therefore trickle onto the screen surface. Alternatively, the aerodynamic nap-forming method according to Paschne, developed at the Institute for Textile Technology at the Rhineland-Westphalia Technical University, Aachen, can also be used.

In order to impart a strength on the non-woven, the non-woven can be doubled.

“Doubling” comprises here a layering of non-wovens on one another. In particular, the two, three or more layers can be connected with one another. Connecting of these layers of non-wovens can take place by needling. In addition, in the case of anisotropic non-wovens, the respective layers can be oriented differently.

In order to ensure an optimum incorporating of the implant, the fibers or non-woven layers can be aligned so that structures occurring in a body are imitated.

In a further development form, the sandwich structure is strengthened partially thermally or mechanically, wherein the strengthening takes place in particular by a needling. Therefore, the two non-woven layers can be connected with one another.

So that the strengthening structure is able to be fixed locally, the strengthening structure can be connected with a portion of the non-woven in a fixing manner by means of a joining technique, in particular sewing, warp knitting, welding, knitting or needling.

So that the implant can be securely applied at the site of implantation, the implant can have a fixing which is configured in particular as a tissue anchor or bone anchor.

In order to facilitate the introduction and anchoring in an intervertebral space, the implant can be rolled, compressed or folded.

So that a tool which is specially produced for the implant is available to the implant surgeons, the problem can be additionally solved by an implant system with a previously described implant and with a deployment instrument associated with the implant, wherein the deployment instrument is able to be used in particular for the direct implanting of the implant into an organism.

Furthermore, the problem is solved by a package which packs a previously described implant and a previously described implant system, wherein in particular the implant or the implant system remains sterile. Therefore, advantageously, for an implant surgeon, an operation-ready implant system can be made available, which is only removed from a sterile package at the time of the operation.

The invention is explained in further detail below with the aid of an example embodiment. There are shown

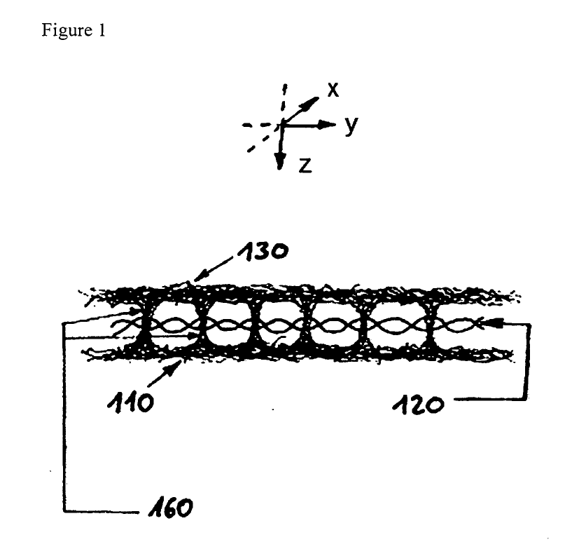

FIG. 1 a diagrammatic sketch of an implant in a cross-sectional view, wherein a two-layer non-woven is needled,

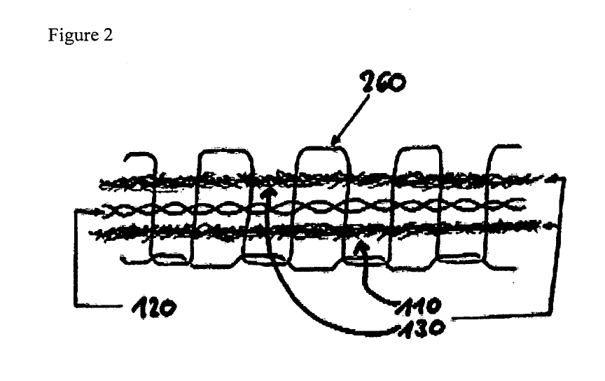

FIG. 2 a diagrammatic sketch of an implant in a cross-sectional view, wherein a two-layer non-woven is provided with a lockstitch seam, and

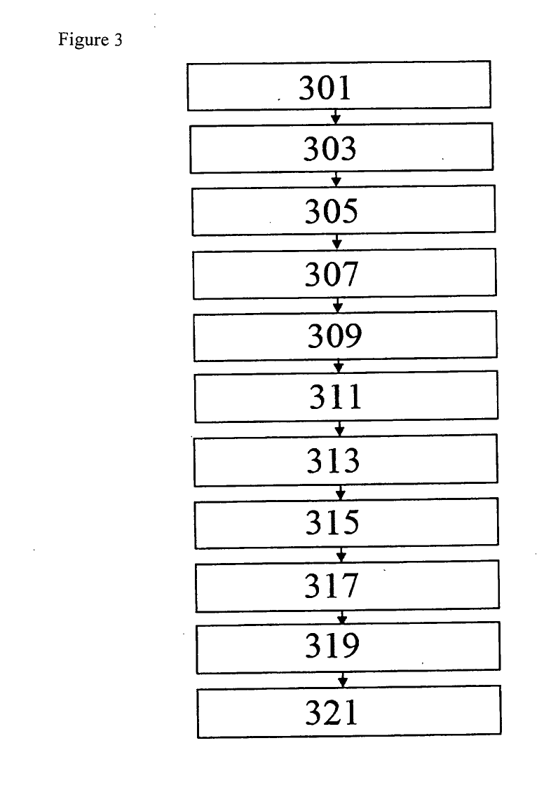

FIG. 3 a flow chart of a basic non-woven production

The implant has three layers: a first non-woven layer 110, a non-resorbable reinforcing structure 120 of polyvinylidene fluoride, which is configured as a knit, and a second non-woven layer 130. The two non-woven layers 110, 130 are connected with one another via connecting means.

In a first alternative, this connection takes place by means of needling, wherein a needling connection 160 is produced. In the needling, fibers of the non-woven are reoriented in Z-direction. The needling takes place here on both sides.

In a second alternative, the connection of the two non-wovens takes place by means of a lockstitch seam 260.

Basically, non-wovens are produced as follows:

The main steps of non-woven production can be divided into the following sub-items (see FIG. 3). Fibers are produced from a polymer 301 (303). These fibers are prepared (305) and then crimped (307). Subsequently, staple fibers are produced. A formation of non-woven 311 takes place by means of the staple fibers 309. A doubling 313 and a strengthening 315 then take place. Finally, a fabrication 317 and the finishing 319 take place, so that ultimately the non-woven 321 is present.

The procedure is as follows for the production of implant non-wovens.

Commercially available polymers are used as multifilaments. These multifilaments are filaments are filaments which consist of several individual threads. The fineness of the filaments is between 1 dtex and 10 dtex [1 dtex=1 g/1000 ml].

In the first step, the fibers are texturized. The texturizing takes place by the knit-deknit method. In this method, firstly a knitting takes place and subsequently a breaking up again of a knitted tube. The stitch size is set here to be such that the emerging crimping of the fibers corresponds to the desired requirements. For knitting, a small circular knitting machine TK83 of the company HARRY LUCAS Textilmaschinenfabrik GmbH & Co. KG is used.

Subsequently, the tube is heat-set in a hot air oven. Here, the material is heated to shortly above the glass transition temperature. In the oven which is used, the heating chamber is flooded with nitrogen as protective gas, so that an oxygen reaction is substantially prevented.

A converting to staple fibers then takes place. Aligned non-wovens are produced by means of the staple fibers. A carding then takes place. In the next step, the non-wovens are needled.

Following this manufacture is the textile-technical analysis of the non-wovens. For this, the surface weights, the thickness, the mechanical characteristics and the pore characteristics are examined. All measurements are carried out under standard climatic conditions in accordance with ISO 139.

The surface weight is determined according to ISO 9073-1. The standard is adapted with regard to the size of the test pieces, because in the present process only non-wovens with a maximum size of 200 cm² are produced. In order to obtain a statistical relevance for the surface weight measurement, the non-woven is cut into three parts, degrees to the production direction. The parts are measured and subsequently weighed with the scales. AE 163; Mettler-Toledo, Ohio, USA.

The thickness of the non-woven is determined according to ISO 9073-2:1995. The standard is adapted as follows. For the thickness measurement, a Universal Micrometer, Frank Prüfgeräte GmbH, Birkenau, is used, which meets the requirements of ISO 9073-2. The ten test pieces (>2500 mm²) required according to the standard are not realized owing to the small overall size of the non-woven. As the data thickness measurement as such is non-destructive, the entire non-woven was used as the test piece and is measured at several locations. For this, the recommended test area of 25 cm² is also reduced to 10 cm². Owing to the smaller test area, a lower test pressure of 0.5 N per cm² is applied.

When the testing of the non-wovens corresponds to the desired parameters, the metallic knit is arranged on the first layer of non-woven. Subsequently, the knit is covered by the second layer of non-woven. Finally, the two non-wovens are needled, so that the implant is present.

1. An implant having a sandwich structure, in particular for promoting a biological closure of the annulus fibrosus of the intervertebral disc, wherein a reinforcing structure is arranged between a two-layer non-woven, wherein the non-woven has in particular a biocompatible material.

2. The implant according to claim 1, wherein the non-woven is resorbable or partially resorbable.

3. The implant according to claim 1, wherein the non-woven has a porosity of 30% to 99%, in particular of 70% to 98%.

4. The implant according to claim 1, wherein the non-woven has one of the following materials or a combination of the materials: a polyglycolic acid, a polyactide acid, a polycaprolactone, a poly-3-hydroxybutyrate, a polydioxanone, copolymers thereof and various derivatives thereof.

5. The implant according to claim 1, wherein the non-woven is enriched with an organic material, in particular a collagen, a hyaluronic acid, a glycosaminoglycan, a demineralized bone particle, a small intestine submucosa preparation and another organic preparation, which has a bioactive factor, in particular TGF-β, FGF, BMP and/or chemoattractants, in particular CXCL 10 and/or XCL 1, and/or is enriched with an inorganic material, in particular a hydroxyapatite, a calcium phosphate, a calcium sulfate, a metal and/or combinations thereof.

6. The implant according to claim 1, wherein the non-woven is cell-colonized, wherein the cell colonization takes place in particular with or without a prior in-vitro tissue maturation, wherein in particular cells with chondrogenic expression potential are able to be used.

7. The implant according to claim 1, wherein a pore size of the non-woven corresponds to one to four times a cell size, so that the cell colonization of the non-woven is optimized, wherein the pore size in particular has a diameter of between 9 μm and 2 mm.

8. The implant according to claim 1, wherein the reinforcing structure is not resorbable or is poorly resorbable.

9. The implant according to claim 1, wherein the reinforcing structure is configured as a knit, a knitted fabric, a weave, a network structure, a twisted yarn, a mesh or a multi-ply fabric.

10. The implant according to claim 1, wherein the reinforcing structure has mono- and/or multi-filaments, in particular forming hernia nets.

11. The implant according to claim 1, wherein the reinforcing structure has one of the following materials or a combination of the materials: a polyvinylidene fluoride, a polypropylene, a polyester, a polyamide, a silk material, a linen material, a metal or derivatives thereof.

12. The implant according to claim 1, wherein the reinforcing structure is enriched with an organic material, in particular a collagen, a hyaluronic acid, a glycosaminoglycan, a demineralized bone particle, a small intestine submucosa preparation and another organic preparation, which has a bioactive factor, in particular TGF-β, FGF, BMP and/or chemoattractants, in particular CXCL 10 and/or XCL 1, and/or is enriched with an inorganic material, in particular a hydroxyapatite, a calcium phosphate, a calcium sulfate, a metal and/or combinations thereof.

13. The implant according to claim 1, wherein the non-woven is produced aerodynamically or mechanically.

14. The implant according to claim 1, wherein the non-woven is doubled.

15. The implant according to claim 1, wherein the fibers or non-woven layers are aligned so that structures occurring in a body are imitated.

16. The implant according to claim 1, wherein the sandwich structure is strengthened at least partially thermally or mechanically, in particular by a needling.

17. The implant according to claim 1, wherein the strengthening structure is connected with a portion of the non-woven in a fixing manner, by means of a joining technique, in particular sewing, warp knitting, welding, knitting or needling.

18. The implant according to claim 1, comprising a fixing, in particular in the form of a tissue anchor or bone anchor.

19. The implant according to claim 1, wherein the implant is rolled, compressed or folded.

20. An implant system with an implant according to claim 1, and with a deployment instrument associated with the implant, wherein the deployment instrument is able to be used in particular for the direct implanting into an organism.

21. A support for an implant according to claim 1, wherein the implant is held in a volume-reduced manner in the support, in particular is folded, rolled up and/or compressed.

22. A use of an implant according to claim 1 for promoting a biological closure of the annulus fibrosus of the intervertebral disc.
